(19) United States
(12) Patent Application Publication
(10) Pub. No.: US 2017/0086684 A1
(43) Pub. Date: Mar. 30, 2017

(54) ELECTRONIC SKIN PATCH FOR HEALTH MONITORING

(71) Applicant: Rowan University, Glassboro, NJ (US)

(72) Inventors: Wei Xue, Glassboro, NJ (US); Xiao Hu, Glassboro, NJ (US); Jeffrey Hettinger, Glassboro, NJ (US)

(73) Assignee: Rowan University, Glassboro, NJ (US)

(21) Appl. No.: 15/278,457

(22) Filed: Sep. 28, 2016

Related U.S. Application Data
(60) Provisional application No. 62/233,741, filed on Sep. 28, 2015.

(51) Int. Cl. A61B 5/026; A61B 5/00; A61B 5/0295; C08K 5/053; C08J 5/18

(52) U.S. Cl. CPC A61B 5/0261; C08K 5/053; C08J 5/18; A61B 5/0295; A61B 5/0022; A61B 5/6832; C08J 2389/00; C08J 2489/00; A61B 2562/12; A61B 2562/16; A61B 2560/0219

(57) ABSTRACT

A wearable electronic system including a base member having an upper surface and a lower surface with the lower surface having a skin-adhering adhesive thereon. At least one sensor is positioned on the upper surface and configured to sense pulse when the base member is adhered to a user's skin. A controller is configured to receive pulse data from the at least one optical sensor and output information representative of a health condition of the user. The base member is manufactured of a flexible, biocompatible material.

### CROSS-REFERENCE TO RELATED APPLICATION

This application claims the benefit of priority under 35 USC §119(e) of U.S. Provisional Patent Application No. 62/233,741, filed on Sep. 28, 2015, the disclosure of which is incorporated herein by reference in its entirety.

### FIELD OF THE INVENTION

This invention relates to an electronic skin (e-skin) patch. More particularly, the invention relates to an electronic skin patch for monitoring health conditions such as heart rate.

### BACKGROUND

The long-term goal of health-related wearable electronics is that a patient's health data can be gathered in real time via body-worn wireless sensors, providing remote, continuous monitoring of human health. This device has the potential to drastically transform the way patients are diagnosed and treated. Among all the heavily research areas in wearable systems, smart textiles have attracted tremendous attention by integrating flexible electronics into daily outfits. Existing technologies rely on electrical or optical measurements. However, the performance of smart textiles is often limited by the gaps between a human body and the fabrics. Furthermore, the smart outfits are usually changed multiple times each week and they are not comfortable to wear during sleep. As a result, the collected subset of possible signals might not represent the actual conditions of the patients.

Systems with longer-term signal collection and minimum human intervention are therefore needed.

### SUMMARY OF THE DISCLOSURE

In at least one embodiment, the present invention provides a wearable electronic system that can be attached to a human body to sense physiological signals such as heartbeat. The patch material is made of protein biopolymers having excellent biocompatibility. The detection is based on optical sensors coupled to a data collection circuit. The measurement in this invention is based on optical sensors that can detect arterial blood oxygen level caused by heartbeats. The patch material is made of protein biopolymers while existing systems (wrist bands and smart watches) use synthetic polymers. The invention results in a smaller and lighter wearable device. The device is a patch on the skin so it is non-obtrusive. The protein biopolymer patch material has better biocompatibility and is more comfortable to wear in comparison with synthetic polymers.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 illustrates a wearable electronic system in accordance with an embodiment of the invention attached to a user's forearm.

FIG. 2 illustrates an exploded view of a wearable electronic system in accordance with an embodiment of the invention.

FIG. 3 illustrates components of an exemplary wearable electronic system in accordance with an embodiment of the invention.

FIG. 4 illustrates exemplary data recorded by a wearable electronic system attached to a user's forearm and to a user's chest.

FIG. 5 is a schematic diagram illustrating a health monitoring system using a wearable electronic skin patch according to some embodiments.

FIG. 6 shows a graph plotting strain vs. stress for various exemplary silk protein substrates.

FIGS. 7A and 7B illustrate the biocompatibility of various proteins, with FIG. 7A showing a graph illustrating the cell viability for various materials and FIG. 7B providing an illustration showing the fibroblasts and neurons on an exemplary material.

FIG. 8A displays the FTIR spectra of the PDMS, pure silk fibroin (SF), and SF-glycerol composite films in the range of 4000-450 cm⁻¹. FIG. 8B shows the FTIR spectra of the pure SF and SF-glycerol composite in the range of 1800-1450 cm⁻¹.

FIG. 9 displays the stress-strain curves of polydimethylsiloxane (PDMS; reference substrate), pure silk fibroin, and silk fibroin containing 20% glycerol plasticizer.

FIG. 10 displays a table showing measurement results of four pulse sensors tested at three different body locations.

FIG. 11 illustrates front, back and side views of a circuit component of a wearable electronic skin patch according to some embodiments.

FIGS. 12A and 12B display the control (no silk film) and optical absorbance of a Mori silk film, respectively; FIG. 12C displays a table showing the optical transmission of three different silk films, sunlight versus the PPG optical sensor light wavelengths.

FIG. 13 illustrates fabrication of a wearable electronic skin patch using nanostructures according to an embodiment.

FIG. 14A displays the standard DSC scans of Mori SF film and Mori SF-glycerol composite film. The samples were heated at 2° C./min from −25° C. to 400° C. with temperature regions related to bound water evaporations (Tw) and sample degradation (Td). FIG. 14B shows the reversing heat capacities of the Mori SF film and Mori SF-glycerol composite film obtained from TMDSC with 2° C./min heating rate, a modulation period of 60s and temperature amplitude of 0.318K.

### DETAILED DESCRIPTION OF PREFERRED EMBODIMENTS

In the drawings, like numerals indicate like elements throughout. Certain terminology is used herein for convenience only and is not to be taken as a limitation on the present invention. The following describes preferred embodiments of the present invention. However, it should be understood, based on this disclosure, that the invention is not limited by the preferred embodiments described herein.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. The term “about” generally refers to plus or minus 10% of the indicated number. For example, “about 10%” may indicate a range of 9% to 11%, and “about 20” may mean from 18 to 22. Other meanings of “about” may be apparent from the context, such as rounding off, so, for example “about 1” may also mean from 0.5 to 1.4.

E-Skin Patch

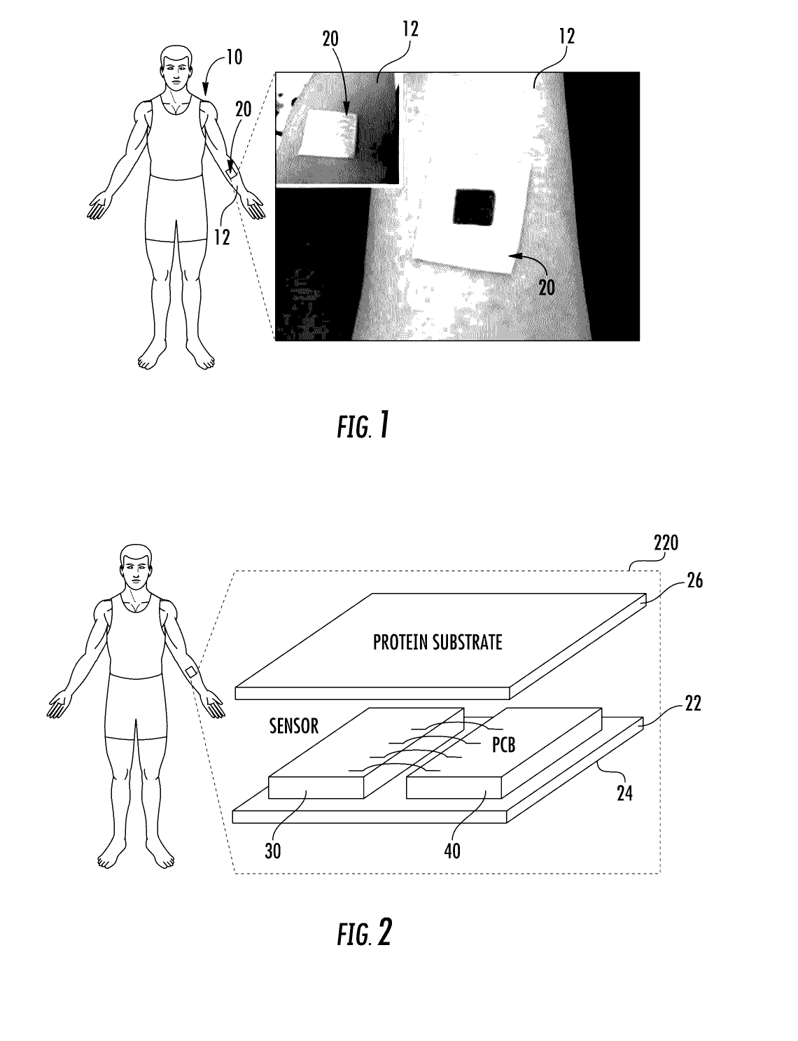

With reference to FIGS. 1-5, in some embodiments, a wearable electronic skin patch 20 is configured to monitor a user's physiological signals in real time. The electronic skin patch 20, when attached to human skin, can continuously monitor the heart rate and respiration rate of a patient in real time. The electronic skin patch 20 utilizes thin electronic sensors, such as piezoelectric or optical sensors, as will be described, to measure the movement of the skin due to a heartbeat, respiration or the like, or the blood flow in the patient, and records the data, for example, in a memory. The recorded data will be stored in an on-chip memory device and later transmitted to a computer or health monitoring system for data processing. Alternatively, the electronic skin patch 20 may include wireless communication capabilities for easy data access by a remote system for use by clinicians from remote sites. Other signals such as movement and temperature can also be monitored by integrating specific sensors into the electronic skin patch.

With reference to FIG. 1, in an embodiment, an electronic skin patch 20 is configured to be adhesively attached to the skin of the user 10, for example, on the forearm 12, although other positions are also contemplated.

With reference to FIG. 2, a wearable electronic skin patch 220 generally includes a top protein substrate 26, a base member 22, which has an upper surface and lower surface, the lower surface may have a skin-adhering adhesive 24 deposited thereon and is used for attaching the electronic skin patch to the skin of a user. Both the top substrate 26 and the base member 22 can be made of polymer materials. The electronic skin patch also includes a sensor device sandwiched between the top protein substrate 26 and the base member 22. In one example, the sensor device may include an electronic health monitoring sensor 30 and a circuit 40 communicatively connected to the monitoring sensor. The health monitoring sensor is configured to generate a sensor signal representative of a health condition of a patient. The circuit will receive the sensor signal, generate or transmit health information based on the received sensor signal to a health monitoring system. The wearable electronic skin patch will be described in detail with reference to FIG. 3.

Sensor and Electronic Components

In FIG. 3, in one embodiment, a sensor device 320 includes an electronic health monitoring sensor 330, and a circuit communicatively coupled to the sensor 330. For example, the circuit may be connected to the sensor via a data bus 45. The circuit may also include a microcontroller, or a processor 342, which is also connected to the data bus 45. Additionally, the circuit may include a memory device 44, a battery 46, and an I/O interface such as USB 49, a communication interface 50, and/or a display 51. Optionally, the battery 46 is a chargeable battery, and the circuit additionally includes a charger 48, which can charge the battery via an external charger. For example, the charger 48 may inductively charge the battery utilizing an external wireless charger.

In an example, the memory can be a non-transitory computer-readable memory that contains data and/or programming instructions. The programming instructions may be programmed to cause the microcontroller to receive sensor signals that are generated by the electronic health monitoring sensor 330, where the sensor signal is representative of a health condition of a patient. The microcontroller can also be programmed to generate health information about the patient based on the sensor signal, store the health information in the memory 44 and/or transmit the health information to a health monitoring system. In one example, the circuit may transmit the health information via I/O interface 49 to an external monitoring device that is connected to the sensor circuit by USB. In another example, the communication interface is a Bluetooth™ interface and the circuit may transmit the health information to a health monitoring system via Bluetooth™. Whether using a wired (e.g. USB) or wireless interface, the electronic skin patch may be periodically connected with an external health monitoring device to transfer health information thereto.

With reference to FIG. 3, the health monitoring sensor 330 can have various designs. For example, the sensor can be an optical sensor, such as a photoplethysmography (PPG) photo sensor that can be used to generate the sensor signal based on the rate of blood flow in the patient. A PPG sensor may be used to measure other health conditions. A PPG sensor generally includes a light source (such as light emitting diode—LED) and a light detecting circuit that can measure a patient's pulse (or hear rate) based on volumetric measurement of the blood flow. For example, most optical sensors used in pulse oximetry use LED as a light source, which travels to arterial blood and reflects back from arterial blood to the photodetector. This requires that the base member material (22 in FIG. 2) to be mostly transparent for the light to go through twice (one original and one reflection). Accordingly, a PPG sensor requires the base member of the electronic skin to be transparent or semi-transparent to let the light through.

In another example, the health monitoring sensor can be a piezoelectric sensor that generally converts vibration into electrical signals, and can be used to measure the movement of the skin to sense vibrations due to heartbeat and respiration, or other health conditions. Piezoelectric sensors may be made of thin film piezoresistor, nanomaterials, carbon nanotubes or nanofibers, to further reduce the size of the device and to provide greater sensitivity of the sensor. Because a piezoelectric sensor detects signals via vibrations, it requires the base member of the electronic skin patch (22 in FIG. 2) that is between the piezoelectric sensor and the skin to be thin and not diminish the mechanical vibration from the pulse, and the piezoelectric sensor should still be able to pick up the vibration with the base member as the intermediate layer.

In one design consideration, the choice of electronic health monitoring sensor may be at least based on the signal-to-noise level of the sensor. For example, with reference to FIG. 10, three different types of piezoelectric sensors and one optical (photoplethysmogram or PPG) sensor, all commercially available, were tested at three locations of the patient's body: the wrist, the forearm and the neck. Among all the tested sensors, the optical sensor provides the highest signal-to-noise level, and can be selected for the electronic skin patch. In comparison, even with mechanical fixtures to apply “pre-pressure” to the skin, the piezoelectric sensors were prone to induced electrical noise. In selecting a suitable optical sensor, according to some design considerations, a commercial available optical sensor is selected, such as model SEN-11574 from http://pulsesensor.com/. The output signal from the sensor during measurement is typically between 0.5-1 V. This is adequate to cover a wide frequency range for reasonable heart beat rates (40 BPM-150 BPM). Alternatively, the sensor may work for heart rates far beyond that range.

In another design consideration, the electronic health monitoring sensor may be a piezoelectric sensor, if, for example, the transparency requirement of the base member of the patch associated with an optical sensor is high and cannot be readily satisfied. The issues with noise associated with piezoelectric sensors may be overcome via extensive signal processing. The processing can be achieved by the microcontroller of the electronic skin patch (e.g. 342 in FIG. 3) or by an external processor which receives the output signals generated by the piezoelectric sensor.

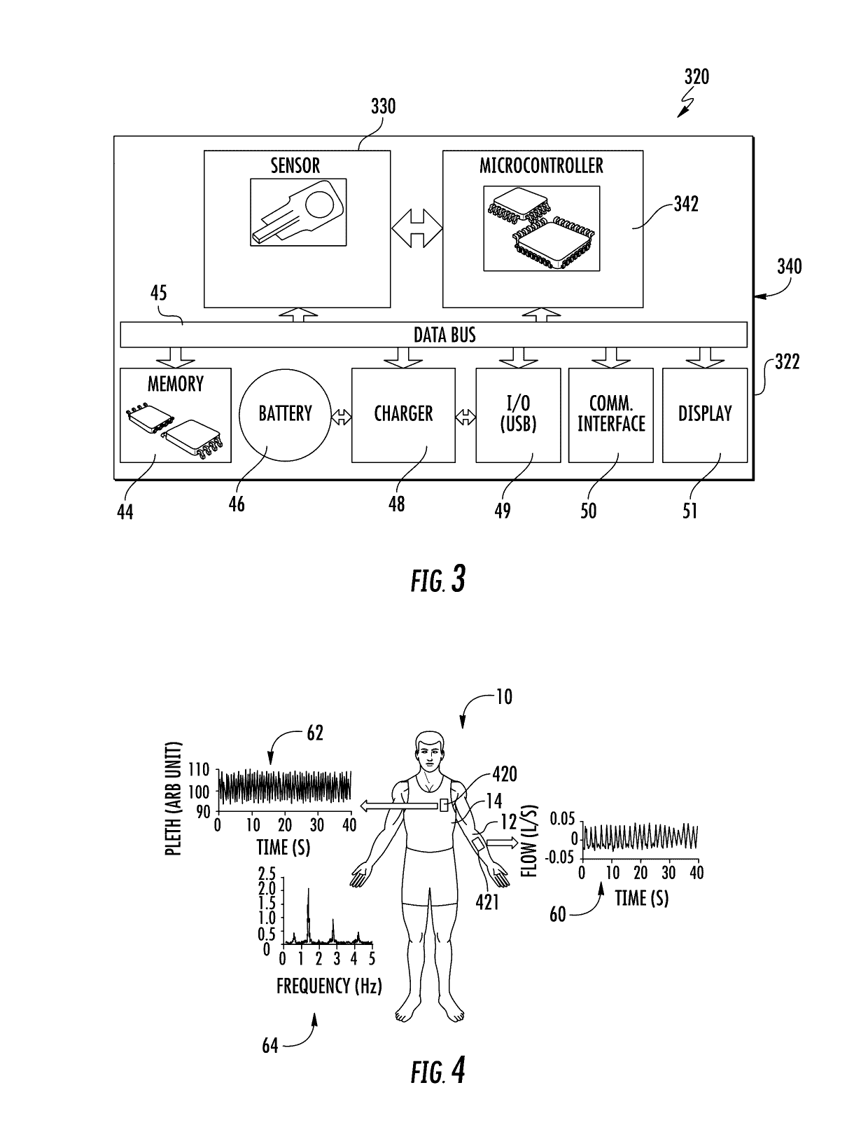

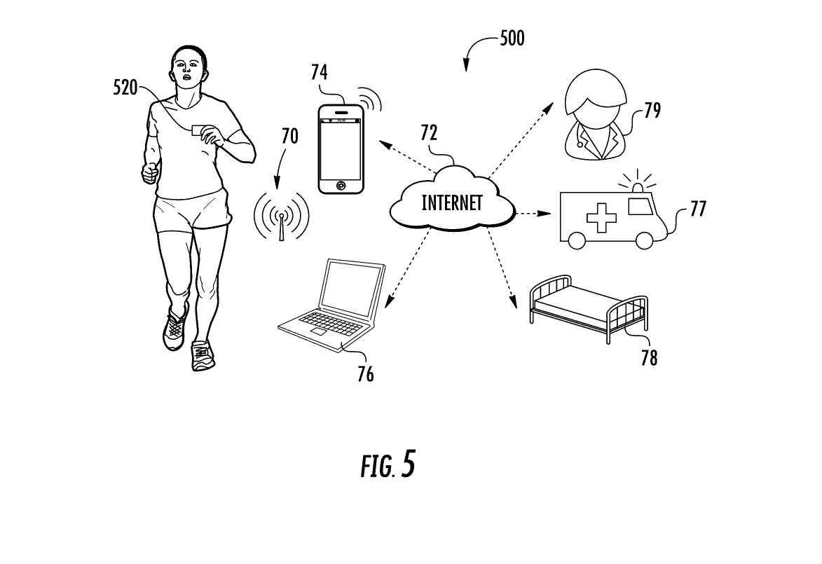

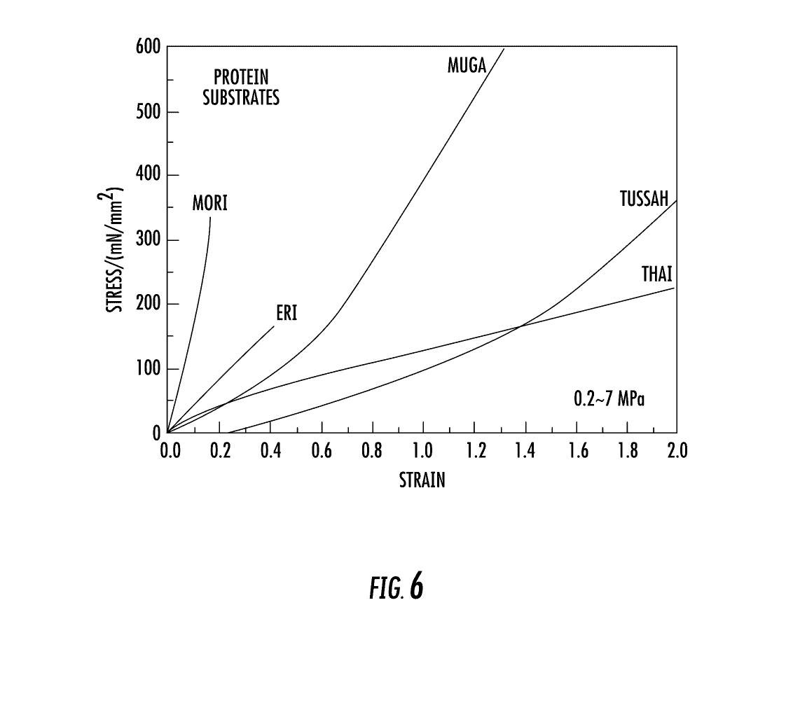

With reference to FIG. 4, the electronic skin patch disclosed in various embodiments in this document may include a piezoelectric sensor and be positioned at various locations on the user 10, which may cause the sensor in the electronic skin patch to sense different vibrations and provide different data. For example, the electronic skin patch 421, is positioned on the user's forearm 12 and generally senses vibration due to blood pulse, thereby outputting data 60 relating to the heart rate, namely the flow rate vs. time. The sensor 420 is positioned on the user's chest 14 and senses vibrations due to a heartbeat and respiration, thereby outputting data relating to respiration rate (plethysmography vs. time) 62 and heart rate (plethysmography vs. frequency) 64. In the event two movements are sensed, the microcontroller of the electronic skin patch can be programmed to utilize signal processing techniques to separate out the data associated with the two movements. In other alternative designs, the sensors are not limited to measuring the identified data and may be configured to sense various other data related to various physiological signals. For example, the heart rate may not be limited to a simple heart rate, but include ECG rhythm of P, Q, R, S, T and U waves.

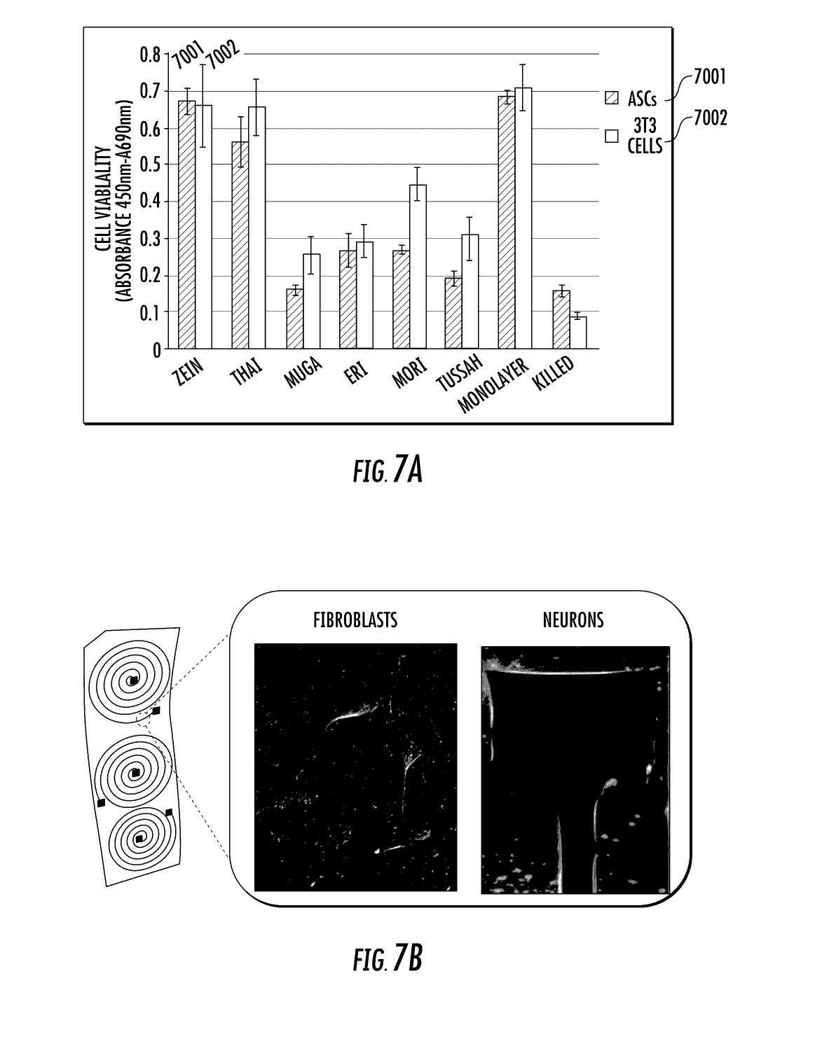

With reference to FIG. 5, in an alternative embodiment, a health monitoring system 500 includes a health monitoring device 76, which contains a processor; a display communicatively connected to the processor; a communication interface communicatively connected to the processor; and a computer-readable medium containing programming instructions, which, when executed, will cause the processor to receive health information from a wearable electronic skin patch 520 via a communication network 70 and display information about a patient's health condition based on the received health information. The wearable electronic skin patch contains atop protein substrate; abuse member having an upper surface and a lower surface with the lower surface having a skin-adhering adhesive thereon; and a sensor device sandwiched between the top protein substrate and the base member.

The sensor device may include an electronic health monitoring sensor that can generate a sensor signal representative of a health condition of a patient. The health monitoring sensor can be a piezoelectric sensor that generates sensor signals from vibrations on the skin of a patent, or an optical sensor, or other known or hereafter sensors that are suitable for measuring a health condition of a patient.

The sensor device will also include a circuit, including a controller, communicatively connected to the electronic health monitoring sensor and configured to receive the sensor signal and transmit health information based on the received sensor signal to the health monitoring device. In one example, the electronic skin patch 520 may have a Bluetooth™ interface and transmit health information to the health monitoring device 76 via Bluetooth™. Alternatively, and/or additionally, the health monitoring device 76 receives the health information from the electronic skin patch 520 and transmits the received health information to a remote mobile device 74 via the Internet 72. Alternatively, and/or additionally, the remote mobile device 74 may communicate directly with the electronic skin patch via Bluetooth™ or other wireless communication protocols. The health monitoring device or the remote mobile device may also be installable or accessible at a hospital room 78, by ambulance personnel 77 or by a treating physician 79.

Biopolymer Materials

The polymeric portion of the wearable electronic skin patch 20 (in FIG. 1) is composed of a flexible, biocompatible material, preferably a biopolymer. For the purposes of the present application the term “biopolymer” comprises any polymer derivable from renewable, natural resources (a so-called “green” material), and the biopolymer itself can be a natural product such as a protein, peptide or polysaccharide. Such biopolymers can include, without limitation, polyhydroxyacids, such as polyglycolic (PGA) acid and polylactic acid (PLA), also known as polylactide; polysaccharides, such as cellulose, chitin and starch; and proteins or peptides, such as corn zein, silk from various silk worm species, such as Bombyx mori, keratin from wool or hair, or collagen or elastin from various tissues; as well as composites or alloys of these. The biopolymers can be made of chiral monomer units, such as lactic acid and amino acids, with the chirality of the monomer units being D, L or D/L (racemic), and the resulting biopolymers can themselves be chiral or racemic, depending on the mixture of specific monomer units. For example, in one embodiment the polylactide is poly-L-lactide (PLLA).

For skin patches the biopolymers are specially designed with the appropriate strength, flexibility, morphology and color to mimic human skin.

Such biopolymers are potentially more biocompatible with human skin than typical synthetic polymers such as latex rubber or vinyl polymers. Such biopolymers also show biodegradability in vivo, and as such can be resorbed by tissues, specifically human tissues.

The protein biopolymers can be plant-derived (for example, without limitation, corn zein and soy proteins or the like), can be insect-derived (for example, without limitation, silk from various species of silkworms, such as the Indian silkworms Antheraea mylitta (Tussah), Philosamia ricini (Eri), and Antheraea assamensis (Muga); the Thailand silkworm (Thai); and the Chinese mulberry silkworm Bombyx mori (B. mori)), or can be higher animal-derived (for example, without limitation, keratin from sheep or llama wool, or hair from other species, including human hair; and collagen or elastin from various tissues) or can include any combinations thereof including synthetic or natural biopolymers. These biomaterials have different protein secondary structures: e.g., alpha-helix structures for zeins, variable beta-sheet structures for silks, and coiled-coils structures for keratins. Therefore, the mechanical properties of the different proteins can be different.

In one embodiment of the invention about 0.5 to about 50 wt % (about 1 to about 45 wt %; about 5 to about 40 wt %; about 10 to about 35 wt %; about 15 to about 30 wt %; about 20 to about 25 wt %; about 10 to about 25 wt %; about 10 to about 30 wt %; about 10 to about 40 wt %; about 10 to about 45 wt %; about 10 to about 50 wt %; about 15 to about 20 wt %; about 15 to about 25 wt %; about 15 to about 35 wt %; about 15 to about 40 wt %; about 15 to about 45 wt %; about 15 to about 50 wt %; about 20 to about 30 wt %; about 20 to about 35 wt %; about 20 to about 40 wt %; about 20 to about 45 wt %; about 20 to about 50 wt %; about 25 to about 30 wt %; about 25 to about 35 wt %; about 25 to about 40 wt %; about 25 to about 45 wt %; about 25 to about 50 wt %; about 30 to about 35 wt %; about 30 to about 40 wt %; about 30 to about 45 wt %; about 30 to about 50 wt %; about 35 to about 40 wt %; about 35 to about 45 wt %; about 35 to about 50 wt %; about 40 to about 45 wt %; about 40 to about 50 wt %; about 0.5 to about 3.0 wt %; or about 0.5 to about 1.5 wt %; or about 0.5 to about 1.0 wt %; or 0.5 wt %; or 1.0 wt %; or 1.5 wt %; or 2.0 wt %; or 2.5 wt %; or 3.0 wt %; or 3.5 wt %; or 4.0 wt %; or 4.5 wt %; or 5.0 wt %) solutions of wild tussah (Antheraea mylitta) silk protein or domesticated mulberry (Bombyx mori) silk protein were separately prepared and was cast onto polydimethylsiloxane (PDMS) or glass substrates to form films. Preferably about 1.0 wt % silk protein solutions were prepared and combined. After drying, the films were removed from the PDMS or glass.

One embodiment of the biopolymer comprises a composite of two or more individual biopolymers. This can be a composite of two or more biopolymers from the same class, such as a mixture of polyhydroxyacids, for example a mixture of PGA and PLA. The composite can also be an alloy of proteins or peptides, such as a mixture of B. mori silk and sheep wool keratin. The composite can also be a mixture of two or more biopolymers from different classes, such as a mixture of B. mori silk and PLA, for example. The ability to readily adjust the composition of the biopolymer allows one to control various tunable biophysical properties of the biopolymer film, and consequently, of the e-skin patch. Such tunable properties include flexibility, elasticity, strength, surface roughness and surface charge.

These proteins can be modified by methods known in organic chemistry to incorporate or remove certain functional groups. In some embodiments the proteins are degummed to remove undesirable components (e.g. soluble silk sericin proteins coated on most silk fibroin fibers) using procedures known in the art (e.g. Rockwood, D. N. et al. Materials fabrication from Bombyx mori silk fibroin, Nat. Protocols. 6, 1612-1631 (2011)).

The protein can be natural, synthetic or recombinant or a combination thereof. For example, the protein can be a natural or recombinant silk protein from different silkworm species.

In some embodiments, two or more proteins are dissolved in a solution to prepare a protein alloy biomaterial. For example, a protein from wild tussah silk and a protein from domesticated mulberry silk can be dissolved in the same solution and processed according to methods of the present invention to obtain a biomaterial of unique mechanical properties. The ratio of two proteins can be about 95:5 to 5:95, or about 90:10 to 10:90, or about 85:15 to 15:85, or about 80:20 to 20:80, or about 75:25 to 25:75, or about 70:30 to 30:70, or about 65:35 to 35:65, or about 60:40 to 40:60, or about 55:45 to 45:55, or about 50:50.

The processed protein exhibits significant differences in properties versus unprocessed protein. For example, a higher crystallinity and/or more organized crystal alignment can be observed in processed protein film. The β-sheet (B) contents in protein films can also be increased from about 1 to about 70% (about 5 to about 60%; or about 10 to about 50%; or about 15 to about 40%; or about 20 to about 35%; or 2%; or 3%; or 4%, etc. up to 70%) after processing. Further, the processed protein may become completely insoluble in water, in contrast to being partially soluble prior to being processed. Other properties including solvent evaporation temperature T_{d1}, degradation temperature T_{d2}, and absorbance may also be changed by processing. In some embodiments, an IR absorbance peak of the processed protein shows a red shift to lower frequency (e.g. 1640 cm⁻¹ shifted to 1620 cm⁻¹). In some embodiments, only a single absorbance peak shows red shift to lower frequency.

The cast silk films tended to be somewhat stiff and brittle in the dry state. However, the e-skin patch is expected to be used in various environments, with high or low humidity levels. Therefore, about 10 to about 35 wt % (based on the weight of the protein component) of one or more environmentally friendly (“green”) biocompatible plasticizers, such as glycerol (glycerin), was blended into the liquid protein compositions so that the cast films can maintain flexibility under different environmental conditions. In one embodiment, the flexibility of the resulting film containing 20 wt % of glycerol was observed to be dramatically increased.

In one embodiment the total amount of plasticizers in the liquid protein composition is about 10 to about 35 wt % versus the protein material; in another embodiment one or more plasticizers are present in a total of about 10 to about 25 wt % versus the protein material; in another embodiment one or more plasticizers are present in a total of about 10 to about 20 wt % versus the protein material; in another embodiment one or more plasticizers are present in a total of about 15 to about 25 wt % versus the protein material. In a preferred embodiment the total amount of plasticizer in the liquid protein composition is about 20 wt % versus the protein material. In other words, the weight ratio of total protein to total plasticizer is about 90:10 to about 65:35; or about 90:10 to about 75:25; or about 90:10 to about 80:20; or about 85:15 to about 75:25. These same ratios apply to the cast films. Without limitation, the biocompatible plasticizers can be selected from sorbitan, sorbitan anhydrides, castor oil, diacetylated monoglycerides, mono- and di-acetylated monoglycerides, triacetin (glycerin triacetate), glycerol (glycerin), erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, polyvinyl alcohol, propylene glycol, triethyl citrate, tributyl citrate, acetyl triethyl citrate, acetyl tributyl citrate, n-butyryl tri-n-hexyl citrate, oleic acid, steric acid, polyethylene glycols, and mixtures of two or more thereof.

The properties of the pure and modified silk film were investigated using various methods. For example, their structures were examined using Fourier transform infrared spectrometry (FTIR, Bruker Tensor 27) and their thermal properties were obtained using temperature-modulated differential scanning calorimetry (TMDSC). FIG. 8A displays the FTIR spectra of the PDMS, pure silk fibroin (SF), and SF-glycerol composite films in the range of 4000-450 cm⁻¹. FIG. 8B shows the FTIR spectra of the pure SF and SF-glycerol composite in the range of 1800-1450 cm⁻¹.

The differential scanning calorimetry (DSC) results can prove the homogeneity of the materials. If the composite is not homogeneous, it will demonstrate both silk composite and pure silk thermal features in the DSC curves indicated by two glass transitions, two melting or degradation peaks. For the inventive composites only one set of glass transition and degradation peaks are observed, which are different from those in the pure silk sample. Therefore, the composite samples are homogeneous, and do not separate into composite and pure silk regions.

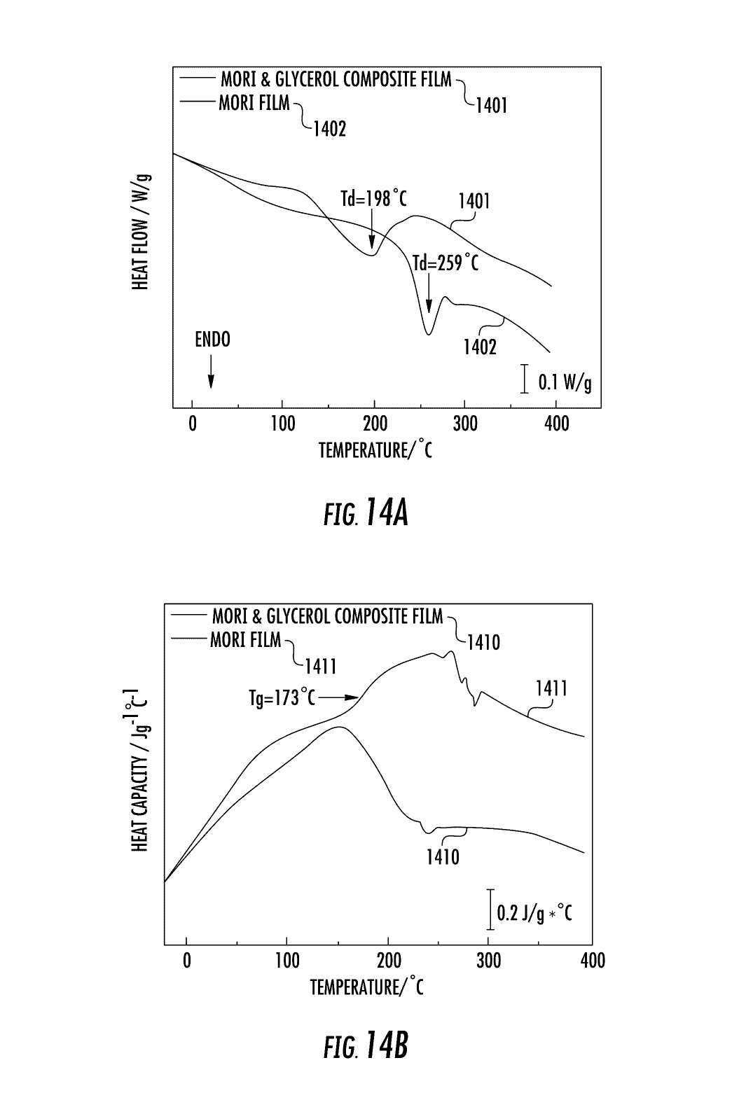

Thus the thermal properties of the SF film and SF-glycerol film samples were examined by standard DSC and temperature modulated DSC (TMDSC). FIG. 14A shows the standard DSC scans of the Mori SF film and Mori SF-glycerol composite film. The Mori silk film showed a small bound water peak around 97° C., and the Mori SF-glycerol composite showed a lower bound water peak around 70° C. After bound water evaporated, both samples showed degradation peaks, at 259° C. for the Mori silk film and at 198° C. for the Mori SF-glycerol composite film, indicating that both samples are thermally stable in the regular temperature range for the present sensor applications.

Temperature modulated DSC was used to measure the reversing thermal properties of the Mori SF film and Mori SF-glycerol film. FIG. 14B shows the reversing heat capacity of both samples. The glass transition region usually appears as a step in the baseline of the recorded DSC curve. A clear glass transition appeared for both samples, at 173° C. for the Mori SF film and at 135° C. for the Mori SF-glycerol composite film, indicating that there will be no significant physical property change for the sensor substrate (either SF or SF-glycerol) when the temperature is below 135° C. The homogeneous glass transition regions for the Mori SF-glycerol composite film also indicated there is no micro-phase separate in the composite film when glycerol content is 20 wt %.

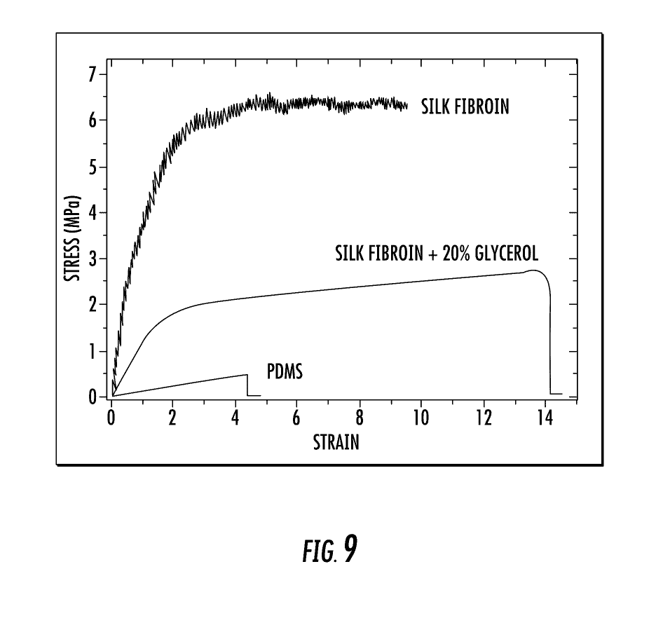

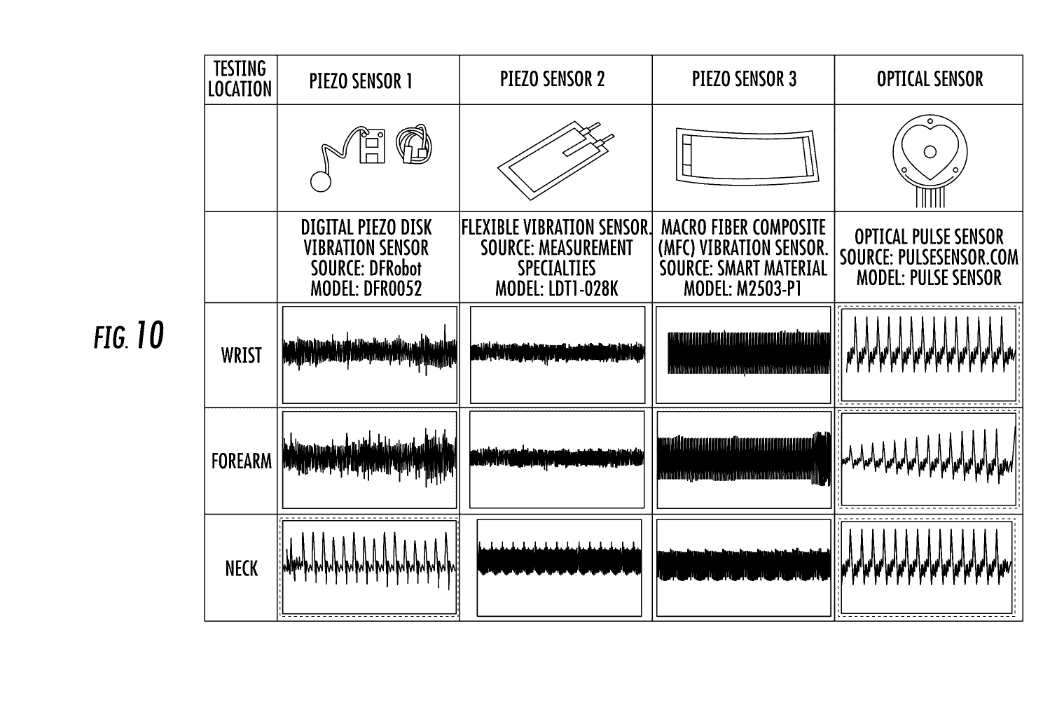

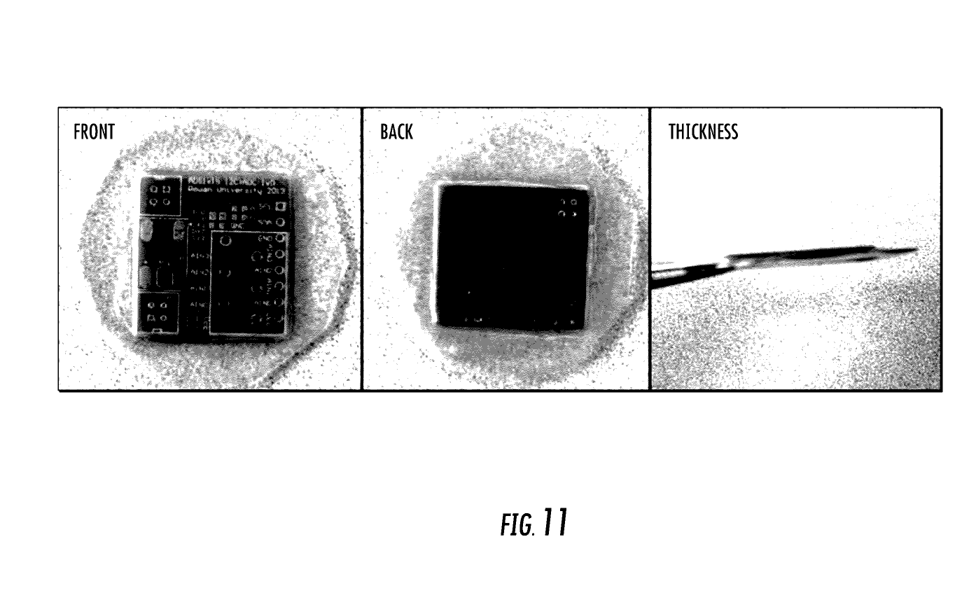

The mechanical properties of silk fibroin, silk-glycerol, and PDMS (as a control substrate) were examined. Using a mechanical tensile testing system the stress-strain data of the three materials were obtained and plotted into one graph for direct comparison, as shown in FIG. 9. There are significant differences of these three materials in terms of their mechanical behavior. The PDMS sample is mostly elastic without showing much plasticity (breaks quickly with no necking effect). In contrast, the stress-strain curve of the pure silk sample (in purple, the highest of the three) shows a large deviation from that of the PDMS. The silk also shows clear elastic and plastic regions with dramatically enhanced strength values in comparison to PDMS.

The silk-glycerol composite sample demonstrates a balanced mechanical behavior in terms of flexibility and strength. The stress-strain curve (the middle curve) of the silk-glycerol composite is between the curves of PDMS and pure silk. It proves that the silk-glycerol composite can survive larger forces and greater elongation without breaking. The silk-glycerol composite is a much softer material compared with the pure silk films. Its mechanical properties make it more suitable for wearable applications. The key parameters are calculated as: Young's modulus of 1.16 MPa, ultimate strength of 2.67 MPa, and ultimate strain of 13.64. Desirably, the Young's modulus should lie in the range 0.26 to 141.11 MPa, the ultimate strength in the range 2 to 36.5 MPa and the ultimate strain before breaking in the range 10 to 71 MPa.

The surfaces and cross-sections of the silk, silk-glycerol composite, and PDMS (reference) films were observed with a scanning electron microscope (SEM). The images disclose the surface microstructures and morphologies of the three materials. No significant changes were found along the depth of the film, indicating homogeneous gel formation. The SEM images of the reference showed a pristine PDMS cross-linked microstructure. PDMS presented no significant change in morphology. The cross-sectional images of all three samples, pure SF, SF-glycerol composite, and PDMS, showed continuous and homogeneous structures without voids. The rough cross-sections indicated the tenacity fracture of the films, which is related to strong mechanical properties.

Integration

Integration of Protein Materials with Circuits:

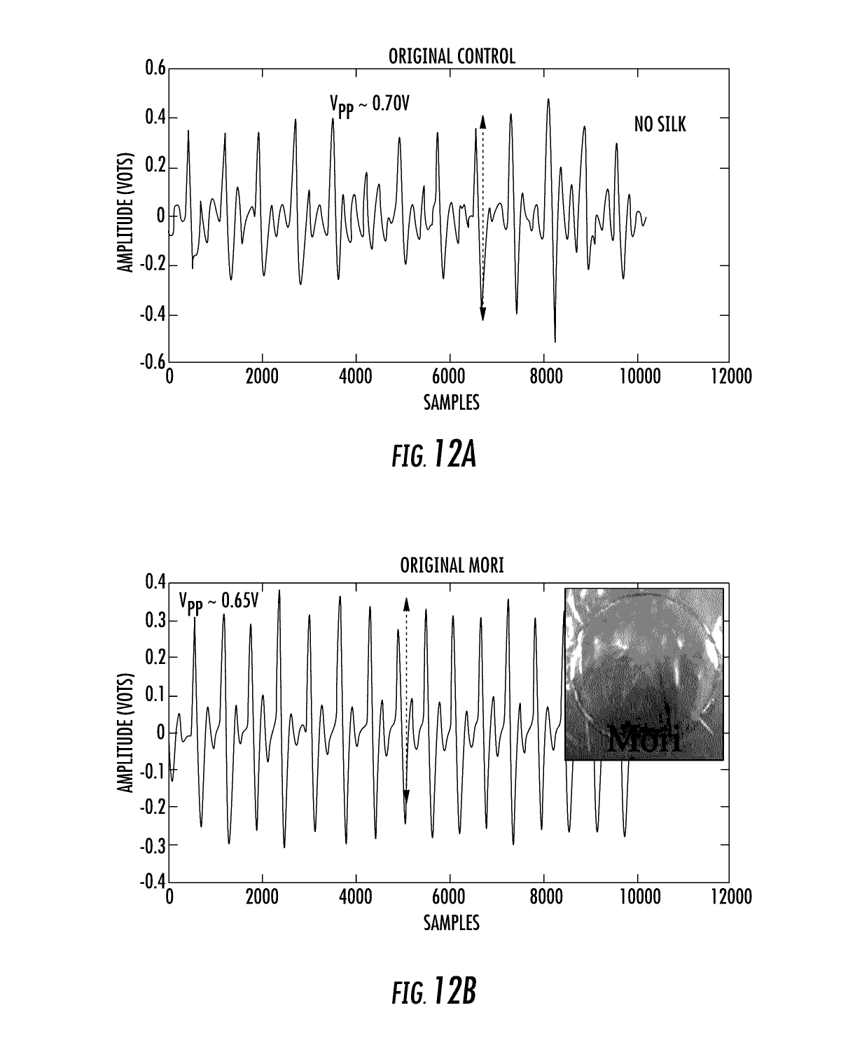

The possibility of integrating protein materials with circuits was explored. Ultimately the current breadboard circuit will be miniaturized onto a small PCB circuit, and will be packaged into protein materials. Initial experiments directed to protein-PCB-protein integration were successful, as shown in FIG. 11. The PCB board shown in FIG. 11 is a bare board without any electronic devices. In an actual PCB circuit, electronic devices will be surface mounted on the board. The circuit will become thicker. The parameters for the integration of silk films will be adjusted accordingly. For example, the quantity of the liquid silk composition will be increased as the PCB circuit will increase in size and thickness.

Integration of Protein Materials with Sensors:

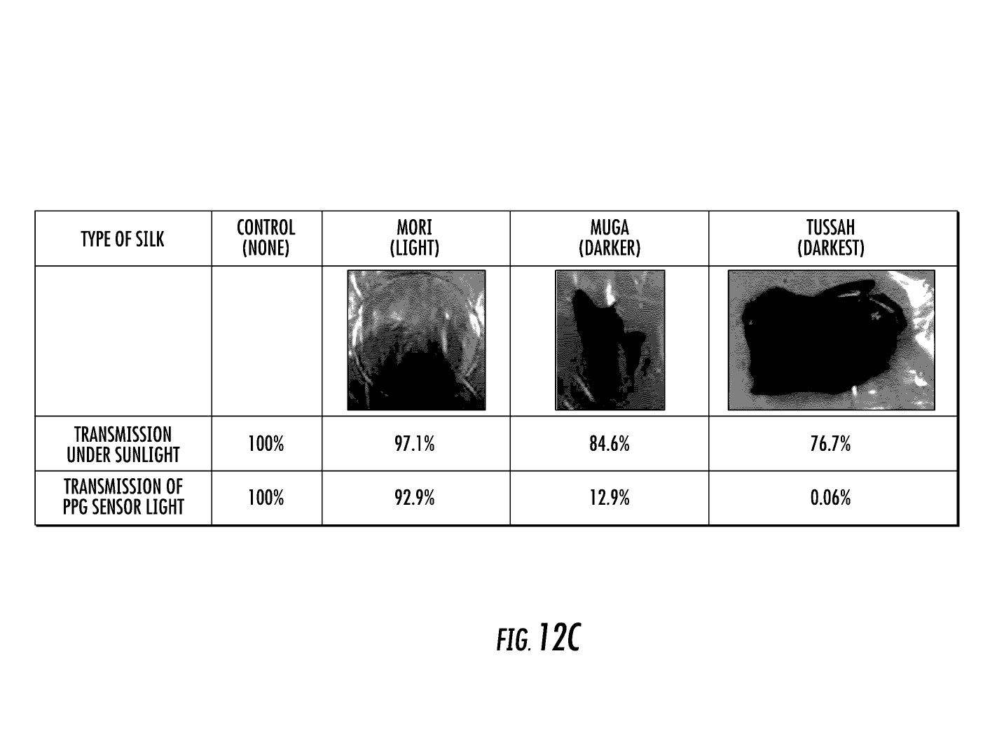

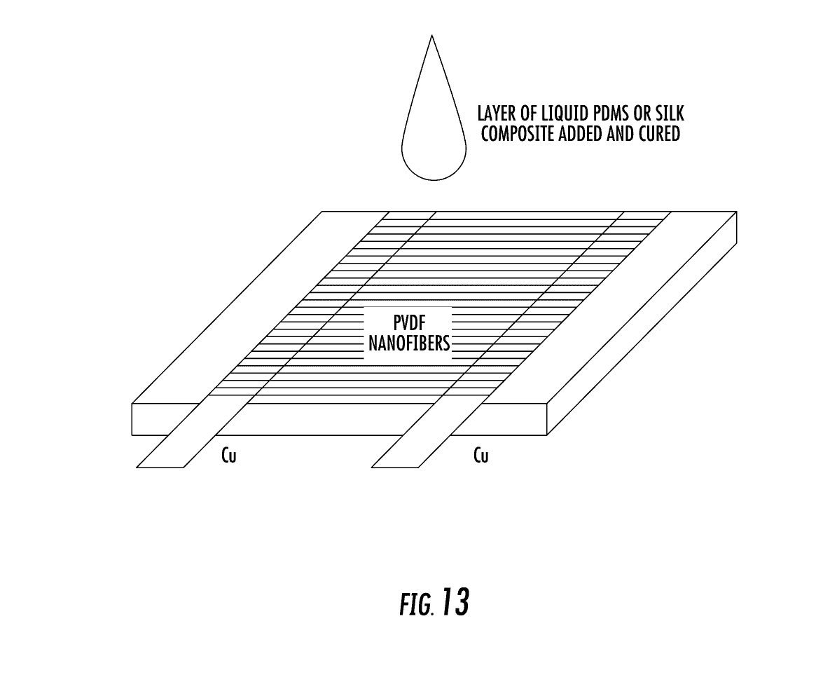

The color of the silk and light transmission properties have an effect on how a PPG sensor reads a pulse. The table in FIG. 12C summarizes the results from the silk-sensor compatibility tests, in which the optical properties, specifically the optical transparency to the wavelengths used by a PPG sensor, of three different silk samples including Mori, Muga, and Tussah, were evaluated. Mori silk was observed to be one of the best performers.

The effect of protein patches on sensors was also tested. The results can help determine a suitable silk material with the desired optical properties. They also help quantify the sensor performance in a silk-packaged system. FIGS. 12A and 12B show the sensor performance through a Mori silk patch, which allows 92.9% transmission of useful signals from the sensor.

Miniaturization of Silk-Based Electronics:

Wearable electronics must be smaller and more portable than in the past. Therefore, further miniaturization of the silk-based e-skin patches is desirable. In one miniaturization embodiment silk substrates have been combined with nanofibers of the functional polymer polyvinylidene difluoride (PVDF), which can convert mechanical vibration into electricity (i.e., serve as a piezoelectric layer) as well as provide flexible support to the patch. A fully flexible device can be created using a pre-cured silk piece as the substrate and liquid silk as the top cover. FIG. 13 shows the integrated device schematic. In this way a device that is approximately 1 cm×2.5 cm has been prepared.

Adhesion with Skin-Friendly Adhesives:

The biopolymer-based patches can be adhered to human skin using skin-friendly adhesives. Suitable examples of such adhesives include, without limitation, acrylic-based adhesives, polyisobutylene-based adhesives and silicone-based adhesives, polyesters or polyurethane-based adhesives. One preferred adhesive is a pressure sensitive acrylate adhesive. Another preferred adhesive is a low-irritation adhesive for sensitive skin.

In certain embodiments the adhesives are also used in fabricating the e-skin patch, for example by layering a biopolymer film, the electronic component and another biopolymer film, with skin-friendly adhesive between these layers to form a sandwich-type patch. In another embodiment, the e-skin patch can have a backing layer of suitable skin-friendly adhesive material that minimizes cutaneous skin reaction, such as a pressure sensitive acrylate adhesive, and is configured such that trauma to the patient skin can be minimized while the patch is worn for the extended period of time (as used herein to include 24, 48, 72 hours or more and up to 120 days). For example, the base member 22 and the top substrate 26 (FIG. 2), if included, are preferably manufactured from a polymeric, biocompatible material, as discussed above.

In another embodiment the electronics can be coated with a water proof material, for example a sealant adhesive such as epoxy or silicone. In some embodiments, the e-skin patch can comprise a medicated patch that releases a medicament, such as antibiotic, beta-blocker, ACE inhibitor, diuretic, or steroid to reduce skin irritation. The e-skin patch can comprise a thin, flexible patch with a polymer grid for stiffening. This grid can be anisotropic and can utilize electronic components to act as a stiffener.

One aspect of the invention is directed to a wearable electronic skin patch for health monitoring, comprising: a top protein substrate; a base member having an upper surface and a lower surface with the lower surface having a skin-adhering adhesive thereon; and a sensor device sandwiched between the top protein substrate and the base member, the sensor device comprising: an electronic health monitoring sensor configured to generate a sensor signal representative of a health condition of a patient, and a circuit, including a controller, communicatively connected to the electronic health monitoring sensor and configured to receive the sensor signal and transmit health information to a health monitoring system; where the electronic skin patch is attachable to skin of the patient continuously for an extended period of time, and where the base member and the top protein substrate each independently comprise a plasticized protein composite film comprising: a) at least one naturally occurring biocompatible protein material selected from the group consisting silks and alloys thereof; and b) at least one biocompatible plasticizer; where the plasticizer is present in about 10% to about 35% by weight. In one embodiment of the electronic skin patch the electronic health monitoring sensor is a photoplethysmography (PPG) photo sensor configured to generate the sensor signal based on the rate of blood flow of the patient. In one embodiment of the electronic skin patch the base member is optically transparent to the wavelengths of light utilized by the PPG. In one embodiment the sensor device further comprises a battery; another embodiment further comprises a charging circuit configured to charge the battery via an external battery charger. In one embodiment the sensor device further comprises a memory configured to store a history of sensor data based on the sensor signals. In another embodiment the sensor device further comprises a wireless communication interface communicatively connected to the circuit and configured to transmit the health information to the health monitoring system wirelessly.

Another aspect of the invention is directed to a health monitoring system, comprising: a health monitoring device comprising: a processor, a display communicatively connected to the processor, a communication interface communicatively connected to the processor, and a computer-readable medium containing programming instructions configured to cause the processor to receive health information from a wearable electronic skin patch via a communication network and display information about a patient's health condition based on the received health information; where the wearable electronic skin patch comprises: a top protein substrate; a base member having an upper surface and a lower surface with the lower surface having a skin-adhering adhesive thereon; and a sensor device sandwiched between the top protein substrate and the base member, the sensor device comprising: an electronic health monitoring sensor configured to generate a sensor signal representative of a health condition of a patient, and a circuit, including a controller, communicatively connected to the electronic health monitoring sensor and configured to receive the sensor signal and transmit health information to the health monitoring device; where the electronic skin patch is attachable to skin of the patient continuously for an extended period of time, and where the base member and the top protein substrate each independently comprise a plasticized protein composite film comprising: a) at least one naturally occurring biocompatible protein material selected from the group consisting silks and alloys thereof; and b) at least one biocompatible plasticizer; where the plasticizer is present in about 10% to about 35% by weight. In one embodiment the electronic health monitoring sensor is a photoplethysmography (PPG) photo sensor configured to generate the sensor signal based on the rate of blood flow of the user. In one embodiment the sensor device further comprises an interface communicatively connected to the circuit and configured to transmit the health information to the health monitoring device wirelessly. In one embodiment the health monitoring device is configured to transmit information about the patient's health condition based on the received health information to a remote mobile device via a communication network.

Another aspect of the invention is directed to a method of fabricating a wearable electronic skin patch for health monitoring, comprising: providing a pre-cured silk substrate comprising: a) at least one naturally occurring biocompatible protein material selected from the group consisting silks and alloys thereof; and b) at least one biocompatible plasticizer; wherein the plasticizer is present in about 10% to about 35% by weight; depositing two or more electrodes on the silk substrate; collecting a PVDF nanostructure between the two or more deposited electrodes on the pre-cured silk substrate to provide a deposited PVDF nanostructure; depositing liquid PDMS or a liquid silk composition on the deposited PVDF nanostructure; and curing the liquid PDMS or silk composition; wherein said liquid silk composition comprises: a) at least one naturally occurring biocompatible silk material selected from the group consisting silks and alloys thereof; b) at least one biocompatible plasticizer selected from the group consisting of sorbitan, sorbitan anhydrides, castor oil, diacetylated monoglycerides, mono- and di-acetylated monoglycerides, triacetin (glycerin triacetate), glycerol (glycerin), erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, polyvinyl alcohol, propylene glycol, triethyl citrate, tributyl citrate, acetyl triethyl citrate, acetyl tributyl citrate, n-butyryl tri-n-hexyl citrate, oleic acid, steric acid, polyethylene glycols, and mixtures of two or more thereof; and c) an aqueous solvent; where the plasticizer is present in the composition in about 10% to about 35% by weight versus the silk material.

Yet another aspect of the invention is directed to a liquid protein composition comprising: a) at least one naturally occurring biocompatible protein material selected from the group consisting of corn zein and silks; b) optionally a naturally occurring biocompatible non-protein material; c) at least one biocompatible plasticizer; and d) an aqueous solvent; where the composition is suitable for casting into films. In one embodiment of the liquid protein composition, the protein material is mori silk (from Bombyx mori). In another embodiment of the liquid protein composition, the biocompatible plasticizer is selected from the group consisting of sorbitan, sorbitan anhydrides, castor oil, diacetylated monoglycerides, mono- and di-acetylated monoglycerides, triacetin (glycerin triacetate), glycerol (glycerin), erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, polyvinyl alcohol, propylene glycol, triethyl citrate, tributyl citrate, acetyl triethyl citrate, acetyl tributyl citrate, n-butyryl tri-n-hexyl citrate, oleic acid, steric acid, polyethylene glycols, and mixtures of two or more thereof, and is present in the composition in about 10% to about 35% by weight versus the protein material.

Another aspect of the invention is directed to a plasticized protein composite film cast from the liquid protein composition disclosed above.

Still another aspect of the invention is directed to a plasticized protein composite film comprising: a) at least one naturally occurring biocompatible protein material selected from the group consisting silks and alloys thereof; and b) at least one biocompatible plasticizer; where the plasticizer is present in about 10% to about 35% by weight. In one embodiment the plasticized protein composite film has a Young's modulus ranging from 0.26 to 141.11 MPa, an ultimate strength ranging from 2 to 36.5 MPa, and an ultimate strain ranging from 10 to 71 MPa. In another embodiment the plasticized protein composite film is characterized as having a homogeneous structure without voids. In another embodiment the plasticized protein composite film is optically transparent to the wavelengths of light utilized by a PPG sensor to be combined with said film to form a wearable electronic skin patch.

### EXAMPLES

### Biopolymer Materials

### Example 1. Silk-Silk Protein Alloy

The method disclosed by Hu et al (Journal of Visualized Experiments, e50891, April 2014, pages 1-13) was used to prepare an alloy of wild tussah silk (Antheraea pernyi) and domestic mulberry silk (Bombyx mori). Briefly, solutions of wild tussah silk protein and domestic mulberry silk protein were prepared and combined in various ratios, which, after appropriate processing and drying, provided variable protein alloy materials useful as the biopolymer components of the e-skin patch.

Various materials were fabricated using this method and analyzed for their morphologies, structures, and properties, versus the synthetic polymer reference PDMS as a control.

### Example 2. Preparation of Silk and Silk-Glycerol Solutions

Silk cocoons came from Bombyx Mori silkworm (China) were boiled for 25 min in an aqueous solution of 0.02 M NaHCO₃ and rinsed thoroughly with Milli-Q water to remove the glue-like sericin proteins. The extracted silk proteins were dried and dissolved into a mixture solution of formic acid and calcium chloride (4 wt % of calcium chloride) for 15 min. After centrifugation and filtration to remove insoluble residues, a 6 wt % SF solution was obtained. To obtain the SF-glycerol solution, glycerol powders were directly added into the SF solution at the mass ratio of 1:4 (glycerol/silk). The final solutions (either SF or SF-glycerol composite) were casted onto pre-cured PDMS substrates to form films. After being rinsed in distilled water for 15 mins, the dried biocompatible SF or SF-glycerol films were obtained.

FTIR spectra of the pristine PDMS, pure SF, and SF-glycerol composite films in the range of 4000-450 cm⁻¹ are shown in FIG. 8A. The spectra for the PDMS film exhibits its typical characteristic IR bands, with absorption at 788-796 cm⁻¹ (—CH₃ rocking and Si—C stretching in Si—CH₃), 1020-1100 cm⁻¹ (Si—O—Si stretching), 1260-1258 cm⁻¹ (CH₃ deformation in Si—CH₃), and 2950-2960 cm⁻¹ (asymmetric CH₃ stretching in Si—CH₃). The SF protein exists mainly in three conformations, namely, random coil, α-helix and β-sheet conformations. The strong absorptions at 1638 cm⁻¹ for amide I (C═O stretching), 1530 cm⁻¹ for amide II (N—H deformation), 1230 cm⁻¹ for amide III (C—N stretching, C═O bending vibration) are observed from the pure SF film group, which indicates that it only contains random coil and α-helical conformations. The spectra of the SF-glycerol composite showed shifts in the absorption band of amide I from 1638 cm⁻¹ (pure silk) to a lower wavenumber (1621 cm⁻¹), reflecting the silk molecules formed insoluble β-sheet structure in the SF-glycerol composite after interlinking with the glycerol molecules (FIG. 8B). The amide II and amide III bands are also shifted to 1526 cm⁻¹ and 1170 cm⁻¹, respectively, illustrating augmentation of the β-sheet structure. The absorption band appearing at 1230 cm⁻¹ (amide III) represents a mixed vibration of CO—N and N—H. Moreover, new absorption bands appears at 1701 cm⁻¹ for carboxylic acids (C═O) in the SF-glycerol composite film, which further verifies the new intermolecular hydrogen bonds among SF molecules.

FIG. 6 shows a graph plotting strain vs. stress for various exemplary silk protein substrates. These curves are similar to those found for different human skins.

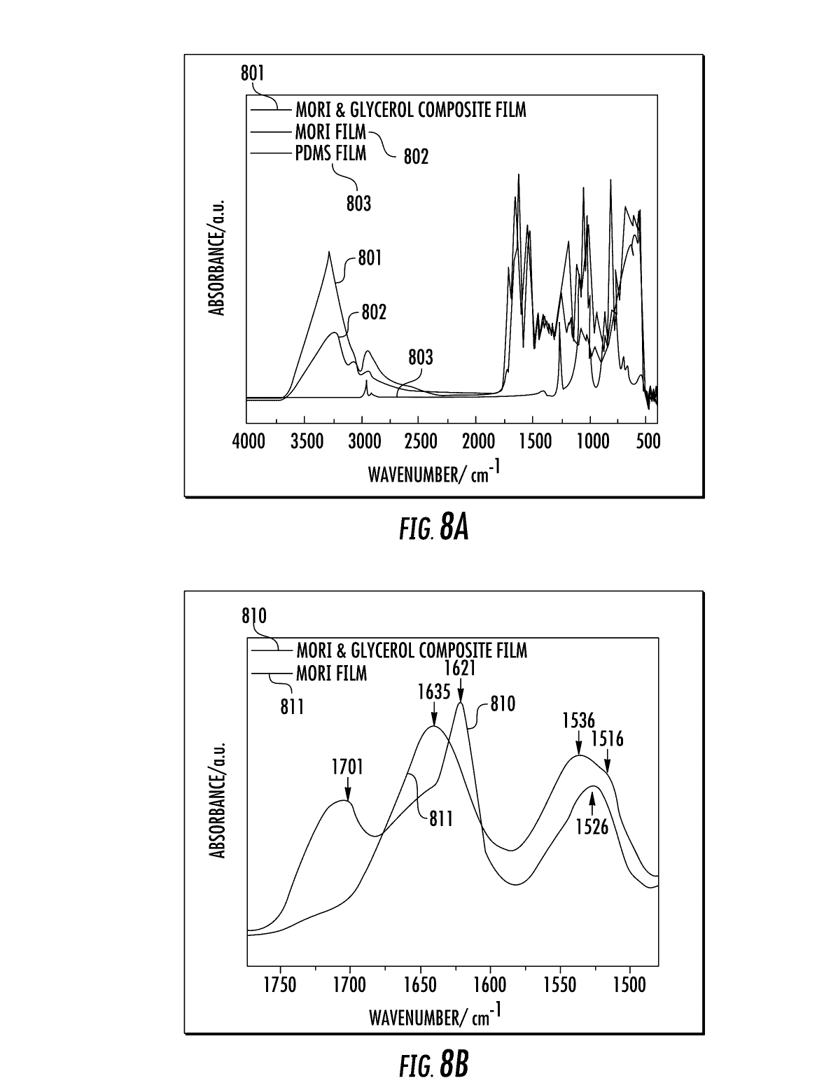

FIGS. 7A and 7B illustrate the biocompatibility of various proteins, with the FIG. 7A graph illustrating the cell viability for various materials and the FIG. 7B illustration showing the fibroblasts and neurons of an exemplary material.

Integration

Our initial trial on the integration was based on a wet pressing technology. The films were first treated by wet streams to soften the surface part of the protein samples, then a 100 N pressing was used on the edge sides of wet films to seal the two films together without using any glue materials. The films were then dried to form a stable two layer structure. Using this technique, a PCB board can be successfully incorporated into a two layer Mori silk films (FIG. 11).

In the illustrated embodiment of FIG. 11, the electronic skin patch is configured to be attachable to the skin of the patient continuously for an extended period of time. This may require special considerations in integrating the electronic sensor and the base member and top protein substrate in fabricating the electronic skin patch so that the integrated package can be miniaturized. For example, the circuit can be miniaturized through (1) careful design of PCB board; (2) selection of smaller chips (microcontroller, Bluetooth™ chips, etc.); (3) selection of multi-functional chips (e.g., microcontroller with large on-chip memory, microcontroller with Bluetooth™ capabilities); and/or (4) moving of uncritical functions from the circuit to remote sites (e.g., remote computer or smartphones for signal processing).

In one embodiment, with reference to FIG. 13, nanofibers can be used to build the electronic skin patch without using a sensor or circuit. In one example, a fabrication process may combine nanofibers with silk films so that the nanofibers are sandwiched between a top and base silk films. The assembly of the device requires both pre-cured and un-cured, liquid silk. The fabrication process includes cutting pre-cured silk into pieces with certain dimensions, such as 45 mm (length)×25 mm (width)×1 mm (thickness), to be used as a base member; placing two electrodes, such as copper strips, horizontally on opposite sides of the silk substrate as electrical leads of the final device. The method further includes collecting nanostructures, such as the PVDF nanofibers, between the two copper strips on top of the silk substrate; placing the device in a tight container and pouring un-cured, liquid silk on the device to cover the entire top surface. The liquid silk could bind seamlessly with the base silk substrate as well as fill in all the cavities of the PVDF nanofiber network. The method further includes drying the materials for 24 hours for the liquid silk to cure.

These and other advantages of the present invention will be apparent to those skilled in the art from the foregoing specification. Accordingly, it will be recognized by those skilled in the art that changes or modifications can be made to the above-described embodiments without departing from the broad inventive concepts of the invention. It should therefore be understood that this invention is not limited to the particular embodiments described herein, but is intended to include all changes and modifications that are within the scope and spirit of the invention as defined in the claims.

What is claimed is:

1. A wearable electronic skin patch for health monitoring, comprising:
a top protein substrate;
a base member having an upper surface and a lower surface with the lower surface having a skin-adhering adhesive thereon; and
a sensor device sandwiched between the top protein substrate and the base member, the sensor device comprising:
an electronic health monitoring sensor configured to generate a sensor signal representative of a health condition of a patient, and
a circuit, including a controller, communicatively connected to the electronic health monitoring sensor and configured to receive the sensor signal and transmit health information to a health monitoring system;
wherein the electronic skin patch is attachable to skin of the patient continuously for an extended period of time, and
wherein the base member and the top protein substrate each independently comprise a plasticized protein composite film comprising:
a) at least one naturally occurring biocompatible protein material selected from the group consisting silks and alloys thereof; and
b) at least one biocompatible plasticizer;
wherein the plasticizer is present in about 10% to about 35% by weight.

2. The electronic skin patch of claim 1, wherein the electronic health monitoring sensor is a photoplethysmography (PPG) photo sensor configured to generate the sensor signal based on the rate of blood flow of the patient.

3. The electronic skin patch of claim 2, wherein said base member is optically transparent to the wavelengths of light utilized by the PPG.

4. The electronic skin patch of claim 1, wherein the sensor device further comprises a battery.

5. The electronic skin patch of claim 4, wherein the sensor device further comprises a charging circuit configured to charge the battery via an external battery charger.

6. The electronic skin patch of claim 1, wherein the sensor device further comprises a memory configured to store a history of sensor data based on the sensor signals.

7. The electronic skin patch of claim 1, wherein the sensor device further comprises a wireless communication interface communicatively connected to the circuit and configured to transmit the health information to the health monitoring system wirelessly.

8. A health monitoring system, comprising:
a health monitoring device comprising:
a processor,
a display communicatively connected to the processor,
a communication interface communicatively connected to the processor, and
a computer-readable medium containing programming instructions configured to cause the processor to receive health information from a wearable electronic skin patch via a communication network and display information about a patient's health condition based on the received health information;
wherein the wearable electronic skin patch comprises:
a top protein substrate;
a base member having an upper surface and a lower surface with the lower surface having a skin-adhering adhesive thereon; and
a sensor device sandwiched between the top protein substrate and the base member, the sensor device comprising:
an electronic health monitoring sensor configured to generate a sensor signal representative of a health condition of a patient, and
a circuit, including a controller, communicatively connected to the electronic health monitoring sensor and configured to receive the sensor signal and transmit health information to the health monitoring device,
wherein the electronic skin patch is attachable to skin of the patient continuously for an extended period of time, and
wherein the base member and the top protein substrate each independently comprise a plasticized protein composite film comprising:
a) at least one naturally occurring biocompatible protein material selected from the group consisting silks and alloys thereof; and
b) at least one biocompatible plasticizer;
wherein the plasticizer is present in about 10% to about 35% by weight.

9. The health monitoring system of claim 8, wherein the electronic health monitoring sensor is a photoplethysmography (PPG) photo sensor configured to generate the sensor signal based on the rate of blood flow of the user.

10. The health monitoring system of claim 8, wherein the sensor device further comprises an interface communicatively connected to the circuit and configured to transmit the health information to the health monitoring device wirelessly.

11. The health monitoring system of claim 8, wherein the health monitoring device is configured to transmit information about the patient's health condition based on the received health information to a remote mobile device via a communication network.

12. A method of fabricating a wearable electronic skin patch for health monitoring, comprising:
providing a pre-cured silk substrate comprising:
a) at least one naturally occurring biocompatible protein material selected from the group consisting silks and alloys thereof; and
b) at least one biocompatible plasticizer;
wherein the plasticizer is present in about 10% to about 35% by weight;
depositing two or more electrodes on the silk substrate;
collecting a PVDF nanostructure between the two or more deposited electrodes on the pre-cured silk substrate to provide a deposited PVDF nanostructure;
depositing liquid PDMS or a liquid silk composition on the deposited PVDF nanostructure; and
curing the liquid PDMS or silk composition;
wherein said liquid silk composition comprises:
a) at least one naturally occurring biocompatible silk material selected from the group consisting silks and alloys thereof;
b) at least one biocompatible plasticizer selected from the group consisting of sorbitan, sorbitan anhydrides, castor oil, diacetylated monoglycerides, mono- and di-acetylated monoglycerides, triacetin (glycerin triacetate), glycerol (glycerin), erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, polyvinyl alcohol, propylene glycol, triethyl citrate, tributyl citrate, acetyl triethyl citrate, acetyl tributyl citrate, n-butyryl tri-n-hexyl citrate, oleic acid, steric acid, polyethylene glycols, and mixtures of two or more thereof; and
c) an aqueous solvent;
wherein the plasticizer is present in the composition in about 10% to about 35% by weight versus the silk material.

13. A liquid protein composition comprising:
a) at least one naturally occurring biocompatible protein material selected from the group consisting of corn zein and silks;
b) optionally a naturally occurring biocompatible non-protein material;
c) at least one biocompatible plasticizer; and
d) an aqueous solvent;
wherein said composition is suitable for casting into films.

14. The composition of claim 13 wherein said protein material is mori silk (Bombyx mori).

15. The composition of claim 13 wherein said biocompatible plasticizer is selected from the group consisting of sorbitan, sorbitan anhydrides, castor oil, diacetylated monoglycerides, mono- and di-acetylated monoglycerides, triacetin (glycerin triacetate), glycerol (glycerin), erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, polyvinyl alcohol, propylene glycol, triethyl citrate, tributyl citrate, acetyl triethyl citrate, acetyl tributyl citrate, n-butyryl tri-n-hexyl citrate, oleic acid, steric acid, polyethylene glycols, and mixtures of two or more thereof, and is present in the composition in about 10% to about 35% by weight versus the protein material.

16. A plasticized protein composite film cast from the liquid protein composition of claim 13.

17. A plasticized protein composite film comprising:
a) at least one naturally occurring biocompatible protein material selected from the group consisting silks and alloys thereof; and
b) at least one biocompatible plasticizer;
wherein the plasticizer is present in about 10% to about 35% by weight.

18. The plasticized protein composite film of claim 17, having a Young's modulus ranging from 0.26 to 141.11 MPa, an ultimate strength ranging from 2 to 36.5 MPa, and an ultimate strain ranging from 10 to 71 MPa.

19. The plasticized protein composite film of claim 17, characterized as having a homogeneous structure without voids.

20. The plasticized protein composite film of claim 17, which is optically transparent to the wavelengths of light utilized by a PPG sensor to be combined with said film to form a wearable electronic skin patch.
